(19) United States
(12) Patent Application Publication
(10) Pub. No.: US 2016/0322210 A1
(43) Pub. Date: Nov. 3, 2016

(54) GLOBALLY OPTIMIZED TARGETED MASS SPECTROMETRY (GOT-MS)

(71) Applicant: UNIVERSITY OF WASHINGTON, SEATTLE, WA (US)

(72) Inventors: Daniel RAFTERY, Seattle, WA (US); Haiwei GU, Seattle, WA (US)

(73) Assignee: UNIVERSITY OF WASHINGTON, SEATTLE, WA (US)

(21) Appl. No.: 15/140,419

(22) Filed: Apr. 27, 2016

Related U.S. Application Data
(60) Provisional application No. 62/155,215, filed on Apr. 30, 2015.

(51) Int. Cl. H01J 49/42; G01N 33/487; G01N 33/493; H01J 49/00; G01N 30/72

(52) U.S. Cl. CPC H01J 49/4215; H01J 49/0045; G01N 30/7233; G01N 33/493; G01N 33/487; H01J 49/0031

(57) ABSTRACT

Globally Optimized Targeted (GOT)-MS, combines many of the advantages of targeted detection and global profiling in metabolomics analysis, including the capability to detect unknowns, broad metabolite coverage, and excellent quantitation. A global search of precursor and product ions using a single triple quadrupole (QQQ) mass spectrometer includes SIM incremental scanning or incremental MS scanning performed in the mass range of 40-2000 Da, followed by tandem MS/MS scanning with incremental collision energy for precursor ions to profile product ions.

This application claims benefit of U.S. provisional patent application No. 62/155,215, filed Apr. 30, 2015, the entire contents of which are incorporated by reference into this application.

### ACKNOWLEDGEMENT OF GOVERNMENT SUPPORT

This invention was made with government support under Grant No. 2 RO1 GM08 291, awarded by the National Institutes of Health. The government has certain rights in the invention.

LENGTHY TABLES
The patent application contains a lengthy table section. A copy of the table is available in electronic form from the USPTO web site (http://seqdata.uspto.gov/?pageRequest=docDetail&DocID=US20160322210A1). An electronic copy of the table will also be available from the USPTO upon request and payment of the fee set forth in 37 CFR 1.19(b)(3).

### REFERENCE TO A TABLE SUBMITTED VIA EFS-WEB

The content of the ASCII text file of the table named “UW58USU1_TABLE2”, which is 94 kb in size was created on Apr. 26, 2016, and electronically submitted via EFS-Web with this application is incorporated herein by reference in its entirety.

### TECHNICAL FIELD OF THE INVENTION

The present invention relates to mass spectrometry that is both globally optimized and targeted, featuring global searching of precursor and product ions. Methods and systems for global profiling, detection, and identification of molecules are provided, including metabolomics.

### BACKGROUND OF THE INVENTION

Metabolomics has emerged as a powerful approach for providing detailed and multilayered information about complex biological processes and systems.¹⁻⁹ Metabolomics studies have resulted in a number of important findings in systems biology and biomarker discovery, including a deeper understanding of cancer metabolism^{10, 11} and drug toxicity,^{12, 13} the potential for improved early disease detection¹⁴⁻¹⁶ or therapy monitoring,^{4,17} as well as applications in environmental science,¹⁸ nutrition,¹⁹ etc. It is clear from these studies and numerous others that significant potential exists for major breakthroughs in metabolomics that will impact many fields.

Mass spectrometry (MS) based methods, specifically global profiling or targeted detection, play an important role in metabolomics for detecting and quantifying metabolites,²⁰⁻²⁸ Due to significant advantages of great selectivity and excellent quantitation, targeted MS using a liquid chromatography triple quadrupole (LC-QQQ) mass spectrometer under multiple reaction monitoring (MRM) mode provides an excellent approach for metabolite profiling. Large targeted MS assays, covering ˜200 metabolites in many important metabolic pathways (e.g., glycolysis, the TCA cycle, etc.), can be constructed and used for metabolomics studies.^{11, 15, 29-34} However, the major limitation of targeted metabolomics is reduced metabolite coverage, often to a small to moderate number of well-known compounds. Specialized knowledge about metabolism is necessary to make good use of highly targeted assays, especially those with a small number of pre-defined targets. There remains a need, therefore, to further develop targeted detection by broadening its coverage, including the detection of unknown metabolites.

### SUMMARY OF THE INVENTION

The invention meets this need and others, by providing a method for subjecting a mixture of molecules to a global search of precursor and product ions with a single mass spectrometer (MS), termed Globally Optimized Targeted Mass Spectrometry (GOT-MS). In one embodiment, the method comprises incremental scanning of a complex mixture to obtain precursor ions, followed by tandem MS/MS scanning with incremental collision energy for precursor ions to profile product ions.

All of these steps CaO be performed on a single triple quadrupole (QQQ) mass spectrometer, and are performed without use of pre--selected standards. The mixture of molecules CaO be a biological sample containing large numbers of unknown molecules, allowing for detection of every molecule the QQQ instrument can detect rather than being limited by pre-selected standards.

In one embodiment, the method comprises performing selected ion monitoring (SIM) incremental scanning or incremental MS scanning using a triple quadrupole (QQQ) mass spectrometer to obtain a large number of precursor ions in a certain mass range from the mixture (e.g,, more than 500 precursor ions). The method further comprises carrying out tandem mass spectrometry (MS/MS) scanning with incremental collision energy (CE) for each selected precursor ion to profile product ions, thereby producing selected pairs of precursor and product ions. The method additionally comprises optimizing the selected pairs of precursor and product ions for different MS parameters. The method can be used for globally detecting one or more metabolites within a complex mixture. In one embodiment, the method is used for detecting over 1,000 MRMs. In another embodiment, the method detects over 1,500 MRMs from a pooled serum sample. In another embodiment, the method detects over 1,800 MRMs from a pooled serum sample.

In some embodiments, liquid chromatography is performed on the mixture prior to the scanning. Examples of liquid chromatography include, but are not limited to, HILIC or reverse phase liquid chromatography (RPLC). The HILIC or RPLC can be used, for example, to separate metabolites in the mixture. Both aqueous extracts and lipid extracts can be used in the method.

In one embodiment, the SIM incremental scanning or incremental MS scanning is performed in the mass range of 40-2000 Da. In a more specific embodiment, the incremental scanning is performed in the mass range of 60-600 Da. In another embodiment, the incremental scanning is performed in the mass range of 600-2000 Da. In one embodiment, the CE is of a voltage of 5 V to 250 V. In another embodiment, the CE is of a voltage of 5 V to 200 V. In some representative specific embodiments, the CE is of a voltage selected from the group consisting of 5 V. 15 V. and 25 V. In one embodiment, the CE is of a voltage selected from the group consisting of 5 V, 15 V, 25 V, 50 V, 100 V. 150 V. 200 V, and 250 V. Examples of mixtures for use with the invention include, but are not limited to, a biofluid mixture, or other biological sample, including tissue, or a cellular mixture. Some more specific examples of a biofluid mixture include a serum sample or a urine sample. Other representative examples of a biological sample include, but are not limited to, blood, plasma, saliva, cerebral spinal fluid, milk, cervical secretions, semen, tissue, cell cultures, and the like. The scanning is performed on the mixture (global scanning), and does not require the use of standards.

The invention additionally provides a Globally Optimized Targeted Mass Spectrometry (GOT-MS) system. In one embodiment, the system comprises a component adapted to receive injections of sample (including a biological sample), a scanning component, wherein the scanning component is capable of performing selected ion monitoring (SIM) incremental scanning or incremental MS scanning; a triple quadrupole spectrometer adapted to perform tandem mass spectrometry (MS/MS) and multiple reaction monitoring (MRM); and a detection means adapted to maximize the number of MRMs that can be detected with each injection of sample. In one embodiment, the component adapted to receive injections of sample is a liquid chromatography component, such as, for example, a hydrophilic interaction liquid chromatography (HILIC) or reverse phase (RP) liquid chromatography component. In a typical embodiment, the scanning component scans in a range of 40 to 2000 Daltons. In one embodiment, the MS/MS employs a collision energy of 5 to 250 Volts. In one embodiment, all components of the GOT-MS system are contained within a single instrument. The system does not require use of standards.

The GOT-MS system of the invention can be used in a method of GOT-MS that comprises preparing an extract of metabolites from a biological sample; and injecting the extract into the GOT-MS system. In one embodiment, the extract is an aqueous extract or a lipid extract.

### DESCRIPTION OF THE DRAWINGS

FIGS. 1A-1B. Flow charts for globally optimized targeted mass spectrometry (GOT-MS). (1A) presents the steps common to various embodiments of GOT-MS, scanning (e.g., SIM incremental or MS incremental) to obtain precursor ions, followed by tandem MS/MS scanning to profile product ions. (1B) presents a typical embodiment in which aqueous extracts are first subjected to liquid chromatography (e.g., HILIC or RPLC).

FIG. 2. Typical HILIC-SIMs in the m/z range of 100-190 from a pooled serum sample in GOT-MS, under A) positive and B) negative ion detection modes.

FIG. 3. The distribution of precursor and product ions in the space of Q1 versus Q3 m/z values, under A) GOT-MS positive ionization, B) GOT-MS negative ionization, C) positive ionization in a large traditional targeted MS assay, and D) negative ionization in a large traditional targeted MS assay.

FIG. 4. The distribution of intraday (n=3) CV values A) for all 1,890 GOT-MS MRMs, B) against the integrated areas of GOT-MS MRMs, C) for the 743 Q-TOF variables present in all the data, and D) against the integrated areas of Q-TOF variables present in all the data.

FIG. 5. A) The PCA score plot (PC1 vs. PC2) of the data collected from CRC and healthy control samples, using a large targeted MS assay, B) the volcano plot of variables used in A), C) the PCA score plot (PC1 vs. PC2) of the 26 GOT-MS MRMs with fold changes>2 and Student's t test P-value<0.05, D) the volcano plot of the 155 GOT-MS variables collected from CRC and healthy control samples, E) the PCA score plot (PC1 vs. PC2) for the 230 Q-TOF variables collected from CRC and healthy control samples, and F) the volcano plot of variables used in E). The dashed red-lines show where P=0.05.

FIG. 6. The general HILIC/RP-LC strategy for GOT-MS.

FIG. 7. Typical RP C18-SIMs in the m/z range of 100-190 from a pooled serum sample under A) positive and B) negative ion detection modes in GOT-MS.

FIG. 8, A) The intraday (n=3) and interday (n=3×3 consecutive days) CVs for amino acids detected by GOT-MS. B) the intraday (n=3) and interday (n=3×3 consecutive days) CVs of amino acids detected by GOT-MS, after normalization to the corresponding isotope labeled (U-13C15N-) internal standards, and C) the linearity (R2) of amino acids from the 5 dilution samples in GOT-MS.

FIG. 9. A) The intraday (n=3) and interday (n=3×3 consecutive days) CVs of amino acids detected by Q-TOF-MS. B) the intraday (n=3) and interday (n=3×3 consecutive days) CVs of amino acids detected by Q-TOF-MS, after normalization to the corresponding isotope labeled (U-13C15N-) internal standards, and C) the linearity (R2) of amino acids detected by Q-TOF-MS using the 5 dilution samples.

FIG. 10. The flow chart of GOT-MS-based metabolomics for CRC diagnosis in this study.

FIG. 11. The PCA score plot (PC1 vs. PC2) for the 155 GOT-MS MRMs collected from CRC and healthy control samples.

FIG. 12. A) SIM scan of m/z 147 in GOT-MS, B) the MS/MS spectrum targeting the ions with m/z 147 at 4.4 min in A). C) MRM scan (147→130.1) of glutamine standard, and D) MRM scan (147→130.1) of lysine standard.

FIG. 13. A) The MS/MS spectrum of lysine standard, and B) the MS/MS spectrum of glutamine standard.

### DETAILED DESCRIPTION OF THE INVENTION

Targeted detection is one of the most important methods in mass spectrometry (MS)-based metabolomics; however, its major limitation is the reduced metabolome coverage that results from the limited set of targeted metabolites typically used in the analysis. The invention provides a new approach, Globally Optimized Targeted (GOT)-MS, that combines many of the advantages of targeted detection and global profiling in metabolomics analysis, including the capability to detect unknowns, broad metabolite coverage, and excellent quantitation, The key step in GOT-MS is a global search of precursor and product ions using a single liquid chromatography triple quadrupole (LC-QQQ) mass spectrometer.

As described in the examples presented below, focused on measuring serum metabolites, we obtained 595 precursor ions and 1,890 multiple reaction monitoring (MRM) transitions, under positive and negation ionization modes in the mass range of 60-600 Da. For many of the MRMs/metabolites under investigation, the analytical performance of GOT-MS is better than or at least comparable to that obtained by global profiling using a quadrupole-time of flight (Q-TOF) instrument of similar vintage. Using a study of serum metabolites in colorectal cancer (CRC) as a representative example, GOT-MS significantly outperformed a large targeted MS assay containing ˜160 biologically important metabolites, and provided a complementary approach to traditional global profiling using Q-TOF-MS. The invention provides new methods via GOT-MS, which expands and optimizes the detection capabilities for QQQ-MS through a novel approach, and can significantly advance both basic and clinical metabolic research.

### Definitions

All scientific and technical terms used in this application have meanings commonly used in the art unless otherwise specified. As used in this application, the following words or phrases have the meanings specified.

As used herein, “a global search of precursor and product ions” and “globally selected” refers to scanning and measuring a broad range of molecules having masses as low as 40 Da and as high as 2000 Da. In one embodiment, the molecules have masses ranging from 60 to 600 Da. “Global” searching is distinguished from “targeted” searching, the latter of which is directed at a limited number of metabolites (typically less than 250), and begins with pre-selected standards that limit what can be detected.

As used herein, “optimizing the selected pairs of precursor and product ions for different MS parameters” refers to varying parameters, such as fragmentor voltage, cell accelerator voltage, and collision energy to optimize the signal for MS detection. A signal is considered optimized if the signal has the highest intensity/area comparing different parameters, and the signal to noise ratio is 3 or greater.

As used herein, to “profile ions” means to determine the m/z ratio of an ion.

As used herein, “a” or “an” means at least one, unless clearly indicated otherwise.

### Methods

The invention provides a method for subjecting a mixture of molecules to a global search of precursor and product ions with a single mass spectrometer (MS), termed Globally Optimized Targeted Mass Spectrometry (GOT-MS). In one embodiment, the method comprises incremental scanning of a complex mixture to obtain precursor ions, followed by tandem MS/MS scanning with incremental collision energy for precursor ions to profile product ions, All of these steps can be performed on a single triple quadrupole (QQQ) mass spectrometer, and are performed without use of pre-selected standards. The mixture of molecules can be a biological sample containing large numbers of unknown molecules, allowing for detection of every molecule the QQQ instrument can detect rather than being limited by pre-selected standards. Unlike traditional targeted methods using MRMs from a limited number of pre-selected metabolite standards, the methods of the invention operate in a data- or information-independent mode to acquire the MRMs, directly from biological samples of interest.

In one embodiment, the method comprises performing selected ion monitoring (SIM) incremental scanning or incremental MS scanning using a triple quadrupole (QQQ) mass spectrometer to obtain a large number (500 or more) of precursor ions in a certain mass range from the mixture. The method further comprises carrying out tandem mass spectrometry (MS/MS) scanning with incremental collision energy (CE) for each selected precursor ion to profile product ions, thereby producing selected pairs of precursor and product ions. The method additionally comprises optimizing the selected pairs of precursor and product ions for different MS parameters. The method can be used for globally detecting one or more metabolites within a complex mixture. In one embodiment, the method is used for detecting over 1,000 MRMs. In another embodiment, the method detects over 1,800 MRMs from a pooled serum sample.

In some embodiments, liquid chromatography is performed on the mixture prior to the scanning. Examples of liquid chromatography include, but are not limited to, HILIC or reverse phase liquid chromatography (RPLC). The HILIC or RPLC can be used, for example, to separate metabolites in the mixture. Both aqueous extracts and lipid extracts can be used in the method. Alternatively, the method can begin with direct infusion of sample.

In one embodiment, the SIM incremental scanning or incremental MS scanning is performed in the mass range of 40-2000 Da. In a more specific embodiment, the incremental scanning is performed in the mass range of 60-600 Da. In another embodiment, the incremental scanning is performed in the mass range of 600-2000 Da. In one embodiment, the CE is of a voltage of 5 V to 250 V. In another embodiment, the CE is of a voltage of 5 V to 200 V. In some representative specific embodiments, the CE is of a voltage selected from the group consisting of 5 V, 15 V, and 25 V. In one embodiment, the CE is of a voltage selected from the group consisting of 5 V, 15 V, 25 V, 50 V. 100 V, 150 V, 200 V, and 250 V. Examples of mixtures for use with the invention include, but are not limited to, a biofluid mixture, or other biological sample, including tissue, or a cellular mixture. Some more specific examples of a biofluid mixture include a serum sample or a urine sample. Other representative examples of a biological sample include, but are not limited to, blood, plasma, saliva, cerebral spinal fluid, milk, cervical secretions, semen, tissue, cell cultures, and the like.

### GOT-MS System

The invention additionally provides a Globally Optimized Targeted Mass Spectrometry (GOT-MS) system. In one embodiment, the system comprises a component adapted to receive injections of sample (such as a biological sample), a scanning component, wherein the scanning component is capable of performing selected ion monitoring (SIM) incremental scanning or incremental MS scanning; a triple quadrupole spectrometer adapted to perform tandem mass spectrometry (MS/MS) and multiple reaction monitoring (MRM); and a detection means adapted to maximize the number of MRMs that can be detected with each injection of sample. The scanning component operates in a data- or information-independent mode to acquire the MRMs, unlike, for example, the scanning used in traditional targeted or pseudotargeted metabolomics. The scanning component is adapted to perform global searching of precursor and product ions, allowing for the detection of more than 1,000 metabolites. In some embodiments, the method can be used to detect over 1,500 metabolites, or about 2,000 metabolites.

In one embodiment, the detection means is adapted to detect over 1,000 MRMs. In another embodiment, the detection means is adapted to detect over 1,500 MRMs. In another embodiment, the detection means is adapted to detect over 2,000 MRMs. In one embodiment, the component adapted to receive injections of sample is a liquid chromatography component, such as, for example, a hydrophilic interaction liquid chromatography (HILIC) or reverse phase (RP) liquid chromatography component,

In a typical embodiment, the scanning component scans in a range of 40 to 2000 Daltons. For aqueous extracts, scanning typically is performed in the range of 60 to 600 Da, while scanning for lipid extracts is typically performed in the range of 600-2000 Da. In one embodiment, the MS/MS employs a collision energy of 5 to 250 Volts. In another embodiment, the MS/MS employs a collision energy of 5 to 200 Volts. In one embodiment, all components of the GOT-MS system are contained within a single instrument.

The GOT-MS system of the invention can be used in a method of GOT-MS that comprises preparing an extract of metabolites from a biological sample; and injecting the extract into the GOT-MS system. In one embodiment, the extract is an aqueous extract or a lipid extract.

### EXAMPLES

The following examples are presented to illustrate the present invention and to assist one of ordinary skill in making and using the same. The examples are not intended in any way to otherwise limit the scope of the invention.

### Example 1

### GOT-MS: Reliable Metabolomics Analysis with Broad Coverage

This Example describes a novel approach, Globally Optimized Targeted (GOT)-MS, which retains the advantages of targeted detection and meanwhile possesses the capacity for broad metabolome coverage. The key step in GOT-MS is a global search of precursor and product ions with a single LC-CMC) mass spectrometer. While there are alternative methods to conduct large-scale MRM experirnents,³⁵⁻³⁹ we expand and optimize the detection capabilities of QQQ-MS through an innovative approach. In this proof-of-principle study, we use aqueous extracts from serum samples to develop the GOT-MS method, and also demonstrate the capabilities of GOT-MS in a trial application of metabolomics, namely to use GOT-MS to differentiate colorectal cancer (CRC) patients from healthy controls. The performance of GOT-MS was compared to two alternative approaches, global and targeted profiling, using the same set of samples, and the results show that GOT-MS compares favorably with these widely used methods.

### Experimental Methods

### Chemicals

Acetonitrile (LC-MS grade) and methanol (LC-MS grade) were purchased from Sigma-Aldrich (St. Louis, Mo.). Ammonium acetate (LC-MS grade) and acetic acid (LC-MS grade) were obtained from Fisher Scientific (Pittsburgh, Pa.). DI water was provided in-house by a Synergy Ultrapure Water System from EMD Millipore (Billerica, Mass.). The metabolite standards in the Northwest Metabolomics Research Center (NW-MRC) internal database (included in Table 2, Example 2 below) were purchased from Sigma-Aldrich or Fisher Scientific, Stable isotope labeled amino acids were purchased from Cambridge Isotope Laboratories (Tewksbury, Mass.).

### Biological Samples

To develop GOT-MS, pooled human serum samples were purchased from Innovative Research, Inc. (Novi, Mich.). For the CRC metabolomics study, serum samples from 18 CRC patients and 20 healthy controls were selected from those involved in our previous studies.^{15,25,40} Table 3 (see Example 2 below) shows the demographic and clinical information for these samples. All serum samples were collected in accordance with the protocols approved by the Indiana University School of Medicine and Purdue University Institutional Review Boards. All subjects in the study provided informed consent according to the institutional guidelines.

### Aqueous Metabolite Extraction

Frozen human serum samples were thawed at 4° C., and proteins were precipitated by mixing 50 μL serum with 250 μL cold methanol, After 20 min incubation at −20° C., the mixture was centrifuged at 14,000 RCM for 20 min. The supernatant was transferred into a clean 2.0 mL Eppendorf vial and then dried under vacuum (Eppendorf Vacufuge). The obtained residue was reconstituted in 400 μL Solvent C (40% Solvent A/60% Solvent B, see details in the LC-MS section) prior to MS analysis. In addition, 5 samples were prepared by reconstituting the metabolite residues from 50 μL serum containing spiked U-13C15N-amino acids, into 200 μL (1:4 dilution), 400 μL (1:8 dilution), 600 μL (1:12 dilution), 1,200 μL (1:24 dilution), and 2,400 μL (1:48 dilution) Solvent C. Table S3 shows the concentrations of spiked U-13C15N-amino acids in the 1:4 dilution sample. Intraday coefficients of variation (CVs) were obtained using the data from samples run three times on the same day, and interday CVs were calculated from the samples run three times each day for three consecutive days.

### LC-MS

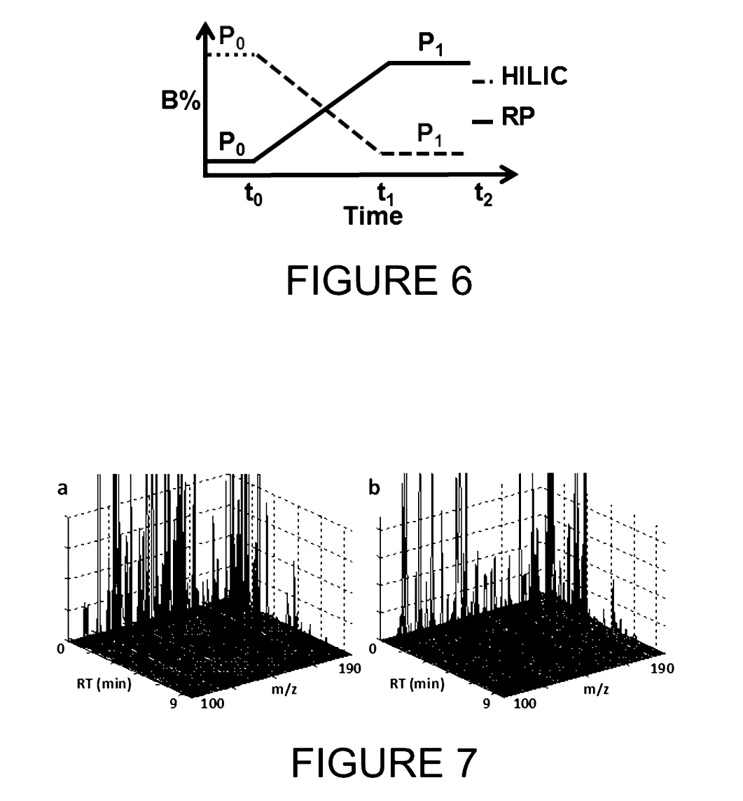

Agilent 1260 LC-6410 Triple Quad MS (Agilent Technologies, Inc., Santa Clara, Calif.): The LC strategy in FIG. 6 is often used in the NW-MRC for both reverse phase (RP) and hydrophilic interaction liquid chromatography (HILIC) separations. After a short (t0) initial isocratic elution (Solvent B, P0), the percentage of Solvent B changes to P1 until t1 and is held at this content until t2. Then the percentage of Solvent B quickly goes back to P0 to prepare for the next injection. In this Example, we primarily used HILIC to separate aqueous metabolites in GOT-MS, employing SeQuant ZIC-cHlLIC columns (150×2.1 mm, 3.0 μm, Merck KGaA, Darmstadt, Germany) at 45° C. Agilent C18 columns (100×3 mm, 1.8 μm, Agilent Technologies, Inc., Santa Clara, Calif.) were used for RP separation at 45° C. In FIG. S1, t0=1 min, t1=6 min, and t2=9 min. Solvent A was 5 mM ammonium acetate in 90% H2O/10% acetonitrile/0.2% acetic acid, and Solvent B was 5 mM ammonium acetate in 90% acetonitrile/1096 H2O/0.2% acetic acid, For HILIC, P0=80%, and P1=30%. For RP, P0=0%, and P1=95%. The flow rate was 0.3 mL/min. 10 μL and 5 μL were injected for negative and positive ionization, respectively. The electrospray ionization (ESI) voltage was 3.8 kV. Adilent MassHunter Qualitative Analysis (version B.07.00) and Quantitative Analysis (version B.07.00) software were used to extract MS peak areas.

Agilent 1200 SL LC-6520 Quadrupole-Time of Flight (Q-TOF) MS (Agilent Technologies, Inc., Santa Clara, Calif.): The separation conditions for the LC-Q-TOF experiments were the same as those for the LC-QQQ (although the LC hardware was not exactly the same). The ESI voltage was also 3.8 kV, and the m/z scan range was 60-1000. The Q-TOF data were extracted using Agilent MassHunter Qualitative Analysis (version 8.07.00), Quantitative Analysis (version 8.07.01), and Mass Profiler Professional (MPP, version B.13.00) software. The absolute intensity threshold for the LC-Q-TOF data extraction was 1000, and the mass accuracy limit was set to 10 ppm.

Agilent 1260 LC-AB Sciex QTrap 5500 MS (AB Sciex, Toronto, ON, Canada): The data of the same 18 CRC samples and 20 healthy controls, collected in our previous study,¹⁵ were used to demonstrate the performance of a traditional large targeted MS assay (with ˜160 metabolites in the detection list). Although not exactly the same, the LC conditions of this assay were very similar to those of the Agilent LC-QQQ or LC-Q-TOF in this study. The experimental methods were detailed in our previous paper.¹⁵

### Multivariate Statistical Analysis

For the CRC study, we performed principal component analysis (PCA) using the PLS toolbox (Version 6.2, Eigenvector Research, Inc., Wenatchee, Wash.) in Matlab (Version T0.4, Mathworks, Natick, Mass.). The data were log10 transformed and mean-centered, prior to PCA analysis.

### Results

### GOT-MS MRMs

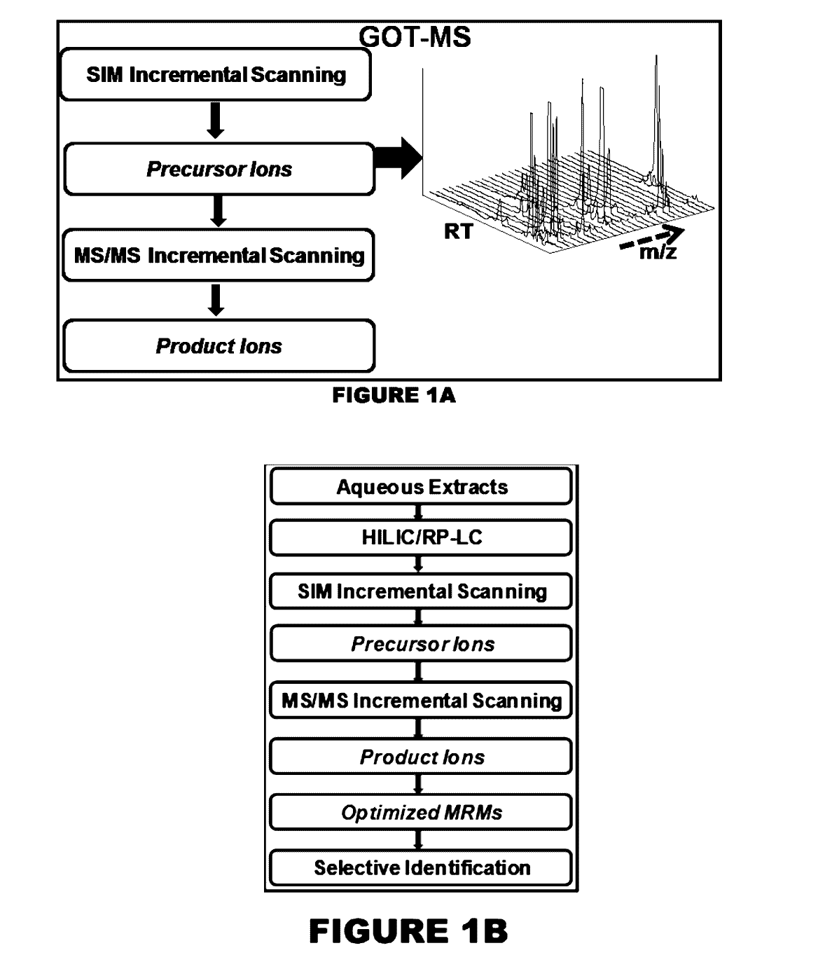

FIG. 1 shows the flow chart for GOT-MS. As it is very challenging, if not impossible, to develop an optimized separation condition for all metabolites, we chose the LC strategy shown in FIG. 6 for both RP and HILIC, HILIC was primarily used to separate aqueous metabolites in the pooled serum samples (1:8 dilution) for GOT-MS development; C18 columns were also used to evaluate whether GOT-MS could be easily adapted to RP separation.

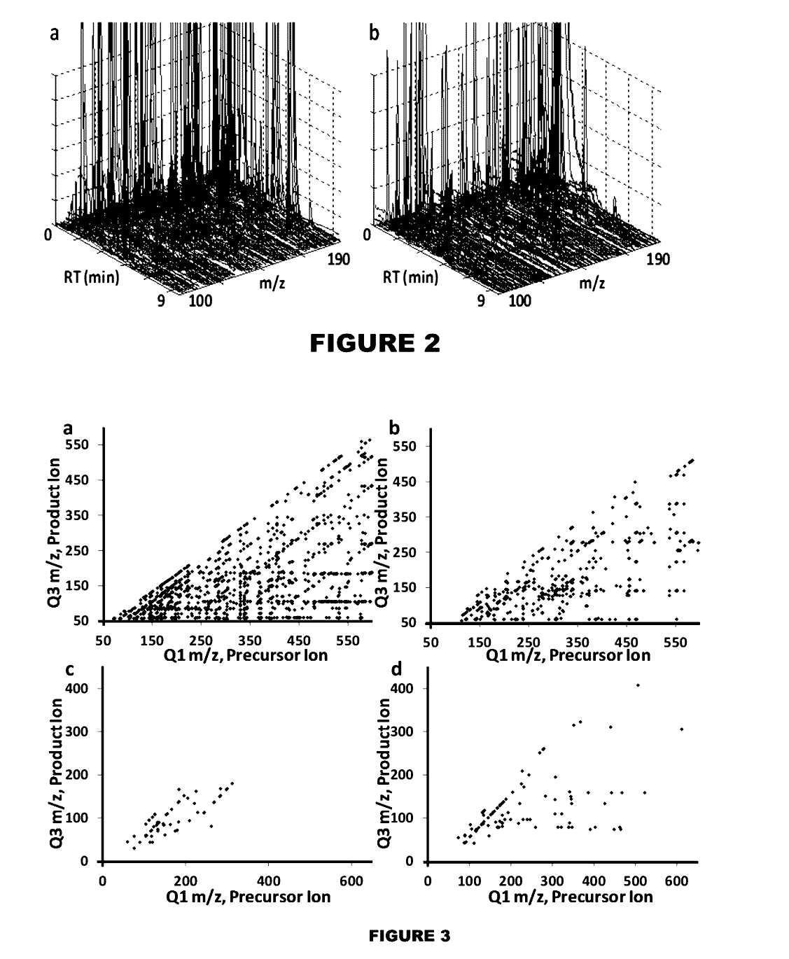

To identify the MRM transitions for GOT-MS, we first performed selected ion monitoring (SIM) incremental scan in the mass range of 60-600 Da in order to cover most of the aqueous metabolites. SIM was employed due to its relatively high sensitivity and good signal-to-noise ratio (S/N). The quadrupole has unit mass resolution; therefore, an m/z increment of 0.5 was used (e.g., m/z: 60, 60.5, 61, etc.). For each injection, an m/z range of 30 (e.g., 60-89.5 Da) was measured (60 SIMs), using a 10 ms scan time (cycle time: ˜0.6 s). We examined each individual SIM, and the m/z values that produced relatively good peak shapes (using manual inspection based on symmetry, peak width, etc.) and S/Ns>3 were selected as precursor ions. As shown in FIG. 2, many HILIC-SIMs were observed in the 100-190 Da mass range from the pooled human serum sample under both positive and negative ion detection. Similarly, FIG. 7 shows that GOT-MS can also produce many SIMs from the same sample, using C18 columns for RP separation. Similar results from urine were also obtained, indicating that GOT-MS can be applied to various kinds of biological samples.

We then carried out tandem mass spectrometry (MS/MS) experiments using product ion scan with different values of the collision energy (CE) to profile product ions. While a wider CE range and different increment values were tested, three CE values were selected in this study: 5 V, 15 V, and 25 V. Most aqueous metabolites fragmented under these CEs. MS/MS spectra using the CE of 5 V provided more accurate m/z values for precursor ions (e.g., 60 could be updated to 60.1). For many precursor ions, different CE values could produce distinct but somewhat overlapping fragmentation patterns, which was helpful to confirm that the MS/MS spectra were from the same metabolite(s). The low m/z limit for MS/MS scan was 40. The number of selected precursor ions was decreased in this step, since the ions with similar m/z values (e.g., m/z 60.1 and 60.5) and the same fragmentation patterns were excluded except for the one with the highest signal intensity.

With both precursor and product ions chosen, ions were detected in MRM mode while optimizing the fragmentor voltage, cell accelerator voltage, and CE. The fragmentor voltage was optimized in the range of 40 V to 180 V using an increment of 20 V, and CE was optimized in the range of 5 V to 40 V with an increment of 5 V. The cell accelerator voltage was evaluated at 2, 4, and 6 V. Finally, 595 GOT-MS precursor ions and 1,890 MRMs were determined from the peaks with reasonable peak shapes (manual inspection) and SiNs>3. Table 2 summarizes all these MRMs. FIGS. 3A and 3B show the distribution of GOT-MS precursor and product ions in the space of Q1 versus Q3 m/z values in positive and negative ion detection modes, respectively. The resulting distribution was quite dense, especially for positive ion mode, since both precursor and product ions were globally searched. Interestingly, a few fragmentation patterns showed visible trends in FIGS. 3A and 3B. For example, many precursor ions lost H2O (M→M-18), and the fragmentation pattern M→M-164 was frequently observed under positive ionization. Several product ions (such as those at m/z 104 and 184) were able to be dissociated from many different precursor ions. In contrast, FIGS. 3C and 3D show the distribution of precursor and product ions in a more traditional large targeted MS assay (targeting ˜160 metabolites) that has been used successfully in the NW-MRC.^{11, 15, 29, 30} It can be clearly seen in FIG. 3 that GOT-MS not only increased the number of precursor ions, but also expanded the Q3 space. This wide coverage can be very important in metabolomics, since different product ions from the same precursor ion can result from different metabolites in complex biological samples.

Scheduling MRM transitions based on retention time (“scheduled MRM mode”) can be used to maximize the number of MRMs in each measurement, Using this approach, we were able to measure all 1,890 GOT-MS MRMs (5 ms scan time for each MRM), in 4 injections (three for positive ions and one for negative ions) using a separation time of 9 min each.

### Analytical Performance

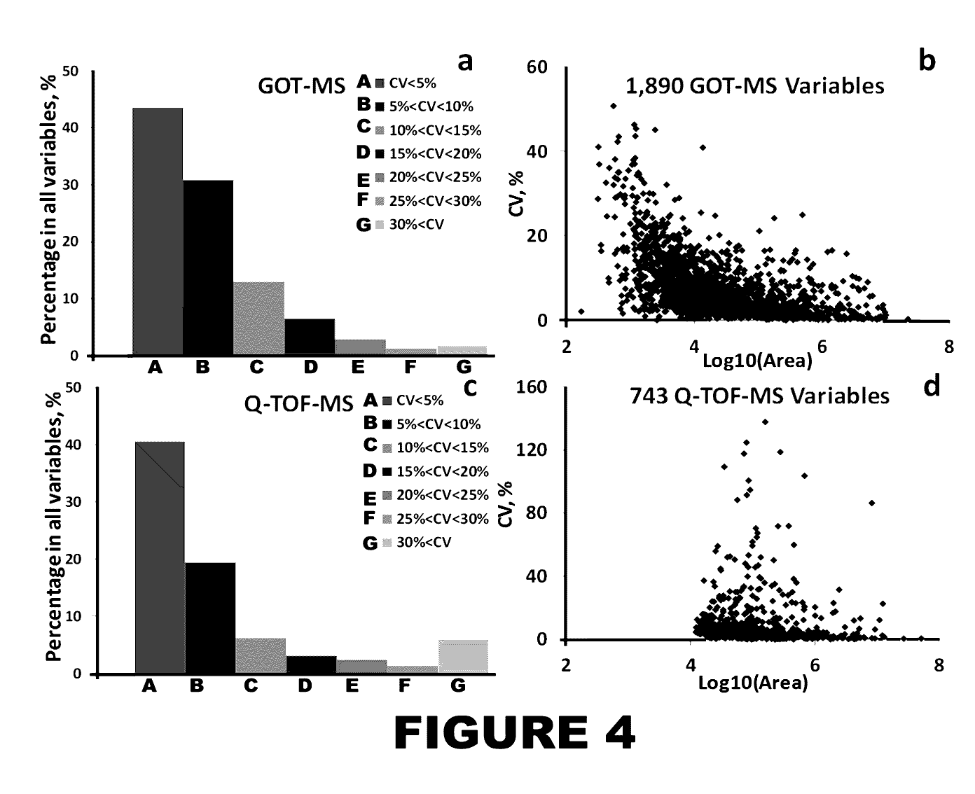

As anticipated, GOT-MS has good quantitative performance, since it is based on an LC-QQQ platform. We examined the intraday CVs (n=3) of all the GOT-MS MRMs. As shown in FIG. 4A, more than 40% of GOT-MS MRMs had CVs<596, and <2% had CVs>30%, indicating excellent reproducibility. Overall, the average CV for GOT-MS detected metabolites was 7.8±7.0%. FIG. 4B shows the relationship between the GOT-MS integrated peak areas and the CV values. The GOT-MS peak areas extended more than 4 orders of magnitude, and the CVs decreased with increased peak area values.

While it is challenging to carry out a perfect comparison between Q-TOF and QQQ instruments, in this study we ran the same samples (1:8 dilution) using essentially the same LC conditions and the Agilent MS systems of similar generation or vintage. FIG. 4C shows the distribution of intraday CV values for 743 Q-TOF variables that were present in all the data (n=3). The reproducibility in FIG. 4C was a little bit worse than that in FIG. 4A, with the average CV of 9.2±16.4%. It was confirmed in FIG. 4D that most of the Q-TOF variables had small intraday CVs, but the CV distribution was obviously more dispersed than that shown in FIG. 4B. The Q-TOF peak areas extended <4 orders of magnitude in FIG. 4D, Notably, while there were 3,471 detected MS features, we selected these 743 variables because of high reliability (n=3), although this number decreased to 500 in 3 days (n=9).

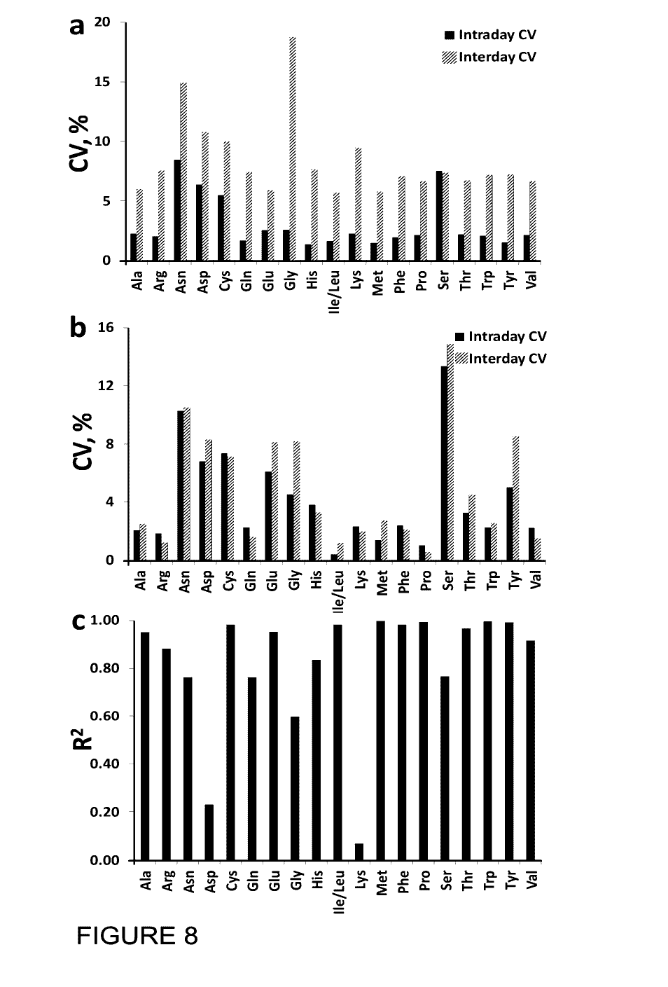

Because analytical detection is compound dependent, we specifically examined amino acids to further compare the performance of GOT-MS and Q-TOF. FIG. 8A shows the intraday (n=3) and interday (n=3×3 consecutive days) CVs of amino acids for GOT-MS. As expected, the interday day average CV (8.3±3.4%) was larger than the intraday average CV (3.0±2.2%). FIG. 8B shows that normalization to internal standards improved the reproducibility, especially the interday average CV (4.8±4.0%). In addition, GOT-MS had good linearity (R2) for most amino acids, based on the data in FIG. 8C collected from the 5 dilution samples.

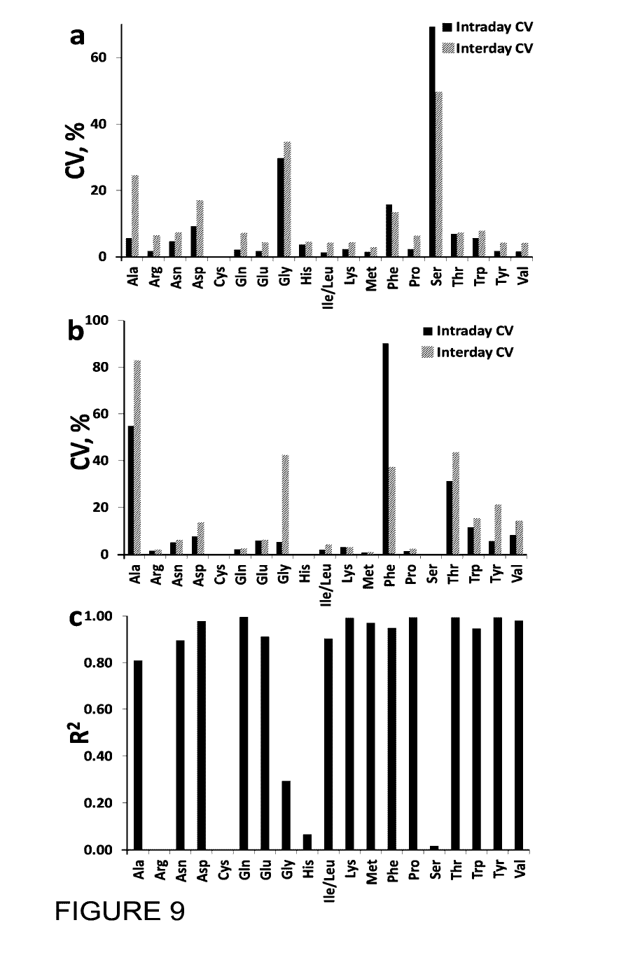

FIG. 9 shows the analytical performance of the Q-TOF instrument for amino acid detection using the same samples. In FIG. 9A, the intraday (9.2±16.6%) and interday (11.7±12.6%) CVs for many amino acids were comparable to those in FIG. 8A, especially for the Q-TOF signals well beyond the detection limit. However, the CVs of a few amino adds with low intensities were very large (and cysteine was not detectable in these samples using the Q-TOF). Surprisingly, normalization to isotope labeled internal standards did not improve the reproducibility of Q-TOF data for many amino acids (FIGS. 8B vs. 9B). This is because the concentrations of spiked internal standards (Table 4) were less than the unlabeled amino acids; U-¹³C¹⁵N-cysteine, U-¹³C¹⁵N-serine, and U-¹³C¹⁵N-histidine were not detectable in these samples using the Q-TOF. As anticipated, the detection limit of the Q-TOF was also worse than that of the QQQ, which caused larger variation in the Q-TOF signals (and contributed to the larger CVs during normalization calculations). For example, we measured the limit of detection (LOD, S/N=3) of valine to be 1×10⁻¹³ mol and 1×10⁻¹¹ mol using the QQQ and Q-TOF, respectively. FIG. 9C shows that the linearity of Q-TOF was good for many amino acids with high intensities. Table 1 summarizes the analytical performance of GOT-MS and Q-TOF-MS.

TABLE 1
Summary of the analytical performance of GOT-MS and Q-TOF
in this study.

| | | Analytical | | | | |
|---|---|---|---|---|---|---|
| | MS Features | Variables | Q-TOF^{a} | | GOT-MS^{a} | |
| Intraday | 1,890 GOT-MS | CVs | | | 7.8 ± | 7.0% |
| (n = 3) | MRMs | Dynamic range | | | >4 orders | |
| | 743 Q-TOF | CVs | 9.2 ± | 16.4% | | |
| | variables present in | Dynamic range | <4 orders | | | |
| | all the data | | | | | |
| | Amino acids^{b} | CVs | 9.2 ± | 16.6%^{c} | 3.0 ± | 2.2% |
| | | CVs after | 14.7 ± | 24.6%^{c,e} | 4.1 ± | 3.4% |
| | | normalization^{d} | | | | |
| | | R^{2f} | 0.76 ± | 0.37^{c} | 0.82 ± | 0.26 |
| Interday | Amino acids^{b} | CVs | 11.7 ± | 12.6%^{c} | 8.3 ± | 3.4% |
| (n = 9 over 3 | | CVs after | 18.5 ± | 22.6%^{c,e} | 4.8 ± | 4.0% |
| consecutive days) | | normalization^{d} | | | | |

^{a}GOT-MS is based on QQQ-MS; the samples were run on Agilent MS systems of similar vintage with essentially the same LC conditions.
^{b}Ile and Leu were integrated together since they had the same MRMs and baseline separation between them was not observed.
^{c}Cysteine was not detectable in these samples using Q-TOF.
^{d}Normalization to the corresponding isotope labeled (U-¹³C¹⁵N-)internal standards was performed.
^{e}U-¹³C¹⁵N-cysteine, U-¹³C¹⁵N-serine, and U-¹³C¹⁵N-histidine were not detectable in these samples using Q-TOF.
^{f}The linearity (R²) of amino acids was obtained from the 5 dilution samples.

### GOT-MS-Based Metabolomics: CRC Biomarker Discovery

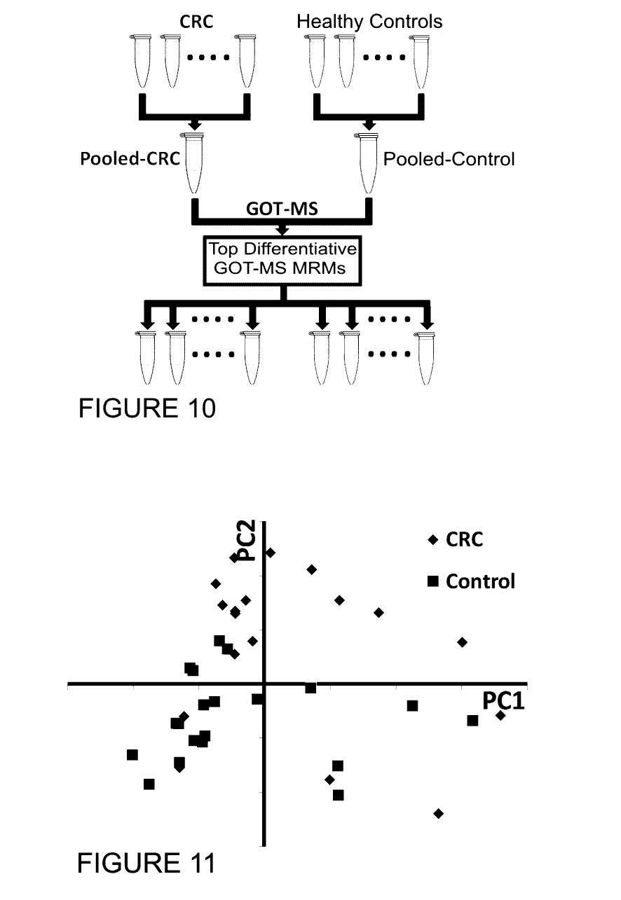

It is important to improve CRC detection, as CRC is one of the most prevalent and deadly cancers in the US and worldwide.⁴¹ To date, many metabolic alterations have been found in CRC tissue,⁴²⁻⁴⁵ serum, ^{15, 46-49} urine,⁵° and fecal water,⁵′ In this Example, we wanted to evaluate the potential for GOT-MS for bioi⁻riarker discovery (e.g., in CRC). We first examined the performance of a traditional large targeted MS assay for differentiating CRC patients (n=18) from healthy controls (n=20).¹⁵ This assay included -160 metabolites from more than 25 metabolic pathways of great biological significance,^{11,15 29, 30} of which 113 metabolites were detected in these serum samples. FIG. 10A shows the PCA score plot/P{}1 vs. PC2), and no clear separation was observed between CRCs and healthy controls, In the volcano plot (FIG. 10B), there was only one variable with fold change (FC)>2 and P<0.05. Although previous studies showed that the combination of multiple serum biomarkers can result in differentiation between cancer and norma1,¹⁵′ ⁴⁶⁴⁹ our results using 18 CRC and 20 healthy control samples suggest that CRC did not cause many very strong perturbations (using the relatively strict criterion Of F{}>2) to major metabolites that are currently often examined.

For analysis using GOT-MS, we used the following approach. To save experimental time, we focused on detecting a moderate number of the possible GOT-MS detected metabolites, rather than focusing on all 1,890 GOT-MS MRMs. Briefly, we mixed CRC and healthy control samples separately, to obtain a pooled sample for each group (Pooled-CRC and Pooled-Control), We then examined all the GOT-MS MRMs on these two pooled samples. We selected the top 93 and 85 GOT-MS MRMs in positive and negative ionization, respectively, according to fold changes (FC>2) and pooled sample-CVs (<20%). The 178 selected MRMs were then run on each individual sample (using two separate injections for positive and negative ions), and finally 155 out of 178 (87%) GOT-MS variables were used in data analysis. FIG. 10 shows the flow chart of this GOT-MS-based rnetabolomics method.

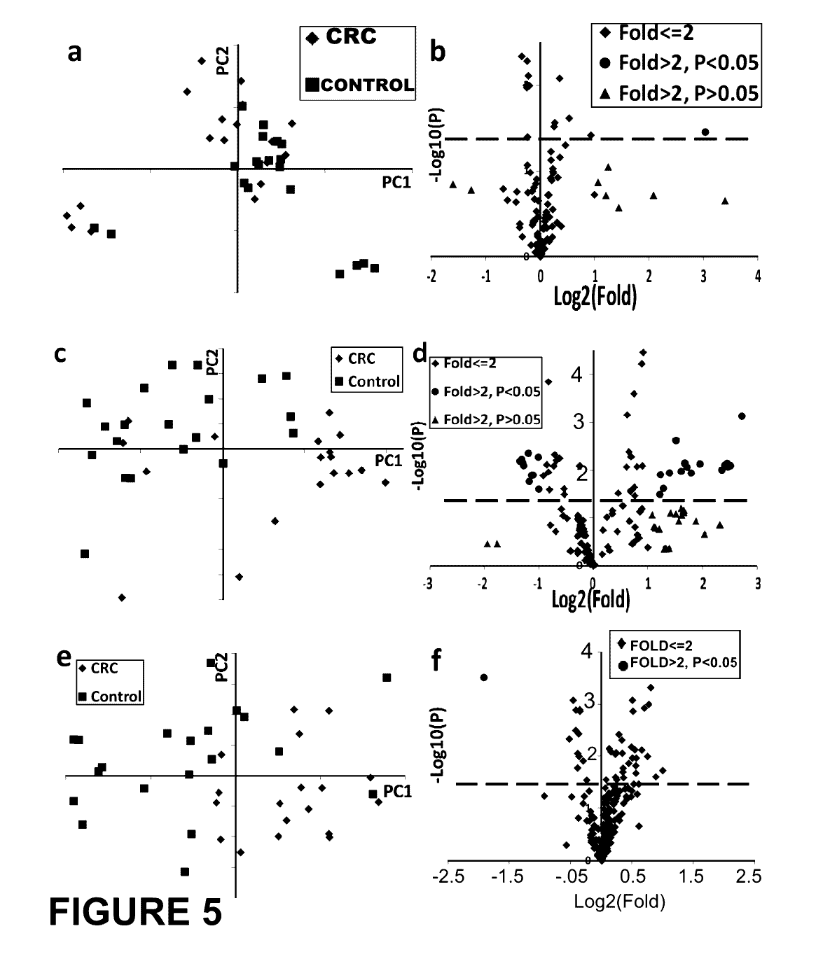

In FIG. 11, the separation between CRCs and healthy controls (mainly along PC2) was better than that observed in FIG. 5A, FIG. 5D shows the volcano plot for the 155 GOT-MS variables. We found 26 variables with FC>2 and P<0.05. We then performed PCA on these 26 important variables, The separation was further improved in FIG. 5c, and the two groups were dearly separated except that 4 healthy samples were mixed with CRC samples.

We then analyzed the same samples using Q-TOF-MS. Traditional global profiling requires sophisticated software for data processing, such as peak picking, peak deconvolution, and peak alignment, Missing values frequently occur in Q-TOF profiling, and 286 (out of 790 in total) variables were present in >80% of the samples in this study, We used the average value of all the samples for data interpolation. Similar to GOT-MS, 230 variables were finally selected for data analysis, after pooled sample-CV (<20%) filtering. In FIG. 5E, the separation between the two groups was better than that in FIG. 5A, but worse than that in FIG. 50 (GOT-MS). Only one variable was found to have FC>2 and P<0.05, as seen in FIG. 5F.

### Preliminary Identification

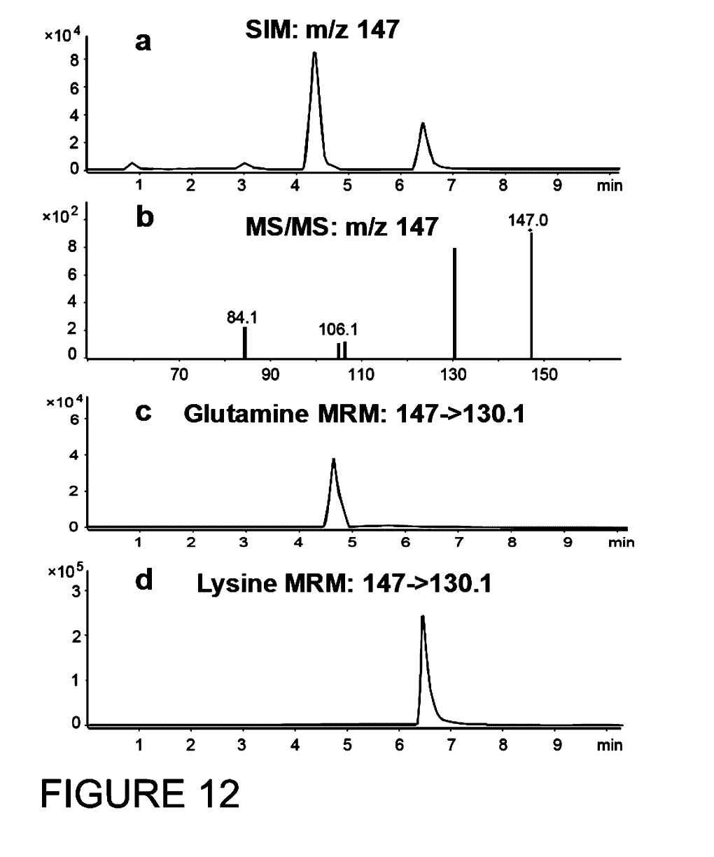

We verified that the identities of -80 GOT-MS MRMs were correct using authentic standards. GOT-MS can of course detect metabolites that are well measured by traditional approaches. As an example, FIGS. 12 (LC retention) and 13 (MS/MS spectra) show the identification of glutamine and lysine (having very similar fragmentation patterns) from the serum GOT-MS results. In addition, we searched each (GOT-MS MRM against the Metlin database,⁵² As shown in Table 2, 770 (out of 1,374) and 183 (out of 516) GOT-MS MRMs could be found in the Metlin database under positive and negative ionization, respectively. We provide up to 5 possible metabolite identities listed in Table 2 for each GOT-MS MRM, from the Metlin database and our own internal database (only product ions with the highest intensities were recorded)^{11, 15, 29, 30} These identities are worth pursuing to further confirm metabolite identification, We acknowledge that it is relatively easy to identify potential metabolites that are contributing to MRM peaks; while, due to many possibilities (such as in-source fragments, isomers, and adducts), it is much more difficult to verify that a particular MRM peak results solely from a specific metabolite, Therefore, we have retained all the GOT-MS MRMs in this Example. For many metabolomics studies we recommend using GOT-MS to obtain the MRMs of special interest first (using statistical filtering, for example), followed by various steps of metabolite identification thereafter,

GOT-MS is also capable to detect unknowns. In fact, 937 (604 in positive mode and 333 in negative mode) GOT-MS MRMs were not found in the Metlin database (Table 2). Although the metabolite identities are unknown, these GOT-MS MRMs should be highly reliable, because of the high selectivity of precursor and product ion pairs that were globally searched. This indicates that GOT-MS is well qualified to detect not only well-known metabolites but also unknowns or less-studied small molecules.

This Example describes a novel approach, GOT-MS, which enables reliable metabolomics analysis with broad coverage. GOT-MS requires only a single LC-QQQ instrument to discover and acquire comprehensive and quantitative MRMs. Data treatment (prior to statistical analysis) is relatively easy, and the data size is small. Although the quadrupole has unit mass resolution, LC retention and fragmentation patterns (MRMs) are useful to resolve metabolites with similar molecular weight. Importantly, the number of precursor ions is limited in a certain mass range by the unit resolution, which is another advantage of GOT-MS.

The GOT-MS approach is different from the newly introduced pseudotargeted metabolomics method that was developed to acquire MRMs without chemical standards.^{35,36} In pseudotargeted metabolomics, a global profiling mass spectrometer, such as Q-TOF, is used to generate pairs of precursor and product ions, and then these MRMs are measured using a QQQ machine. While pseudotargeted MS has been successfully applied in metabolomics, it is not a global approach, since it uses the auto MS/MS or data- or information-dependent mode to acquire the MRMs, which limits the number of MRMs (generally <5 at each moment). It also requires two (expensive) MS platforms, a Q-TOF-MS for identifying MRMs and a QQQ-MS for detection. It is best if the two instruments come from the same manufacturer. In a separate approach, Nikolskiy et al. developed a computational method that is helpful to conduct large-scale MRM experiments: however, this method is dependent on databases (less specific to samples) and is unable to detect unknowns.³⁷ This approach has not been implemented experimentally to date.

The results for the many detected MRMs indicate that the analytical performance of GOT-MS is comparable to or is better than that of a Q-TOF instrument for both global profiling and targeted detection (using instruments of similar vintage). This improved performance results primarily from the fact that GOT-MS is based on the QQQ-MS platform, which was also observed in previous studies.^{35, 36} In addition, while our Q-TOF generally has good reproducibility for strong signals, these signals cannot be too large, because signal saturation will deteriorate mass accuracy (as well as linearity). Therefore, the advantages of high resolution and high mass accuracy can be compromised in traditional global profiling.

In the proof-of-principle study focused on biomarker discovery in CRC, GOT-MS provides a significant improvement over a large targeted MS approach and provides supplementary information to that of traditional global profiling. Traditional targeted detection is limited by databases or pre-defined metabolites (that may not be detectable in the samples), which makes it unable to discover unknowns, GOT-MS removes this bottleneck through a global search of precursor and product ions, using the samples under investigation. Meanwhile, GOT-MS can serve as a valuable supplementary approach to traditional global profiling, because GOT-MS is not dependent on mass resolution/accuracy. The improved reliability of GOT-MS is observed in this example of comparing CRC vs. normal, where GOT-MS analysis resulted in 26 MRMs/metabolites with fold changes>2 and P<0.05, compared to only 1 for the Q-TOF and 1 for the traditional large targeted assay. While the multivariate analysis using the Q-TOF data was successful in separating the two classes of samples, this result was dependent on a large number of metabolites that individually had relatively poor performance. This situation is well-known in the metabolomics field, and makes the job of choosing metabolites for further validation difficult and often not very successful.

Currently, the major limitation of GOT-MS is the required time to optimize the many MRMs for a particular sample type. Using a faster and more sensitive instrument will allow the measurement of more GOT-MS MRMs and in principal could reduce the development time to ˜1 week/sample type (using scheduled experiments). In addition, the example illustrates significant efforts to improve metabolite identification in GOT-MS, Finally, a software package can be developed to automate the whole GOT-MS development process, making GOT-MS more user-friendly to the metabolomics community.

In summary, this Example describes a new MS approach, GOT-MS, which integrates many advantages of traditional global profiling and targeted detection, including the capability to detect unknowns, wide metabolite coverage, and excellent quantitation. GOT-MS was designed to essentially optimize the detection performance of a single LC-QQQ mass spectrometer for broad metabolome coverage, and given the capabilities of QQQ-MS, it is very selective and highly reliable for quantitative analysis. The precursor and product ions are globally searched, resulting in the maximum number of MRMs/metabolites that can be measured by LC-QQQ. GOT-MS has wider metabolite coverage than traditional targeted detection, and provides a valuable supplementary approach to traditional global profiling. Faster and more sensitive LC-QQQ instruments will allow further improvements in speed and coverage for GOT-MS.

### Example 2

### Additional Information

Additional information supporting the study described in Example 1 is presented in he following tables and FIGS. 6-13. The list of GOT-MS MRMs from Example 1 are presented in Table 2. A few precursor and product ion pairs had similar molecular weight, but they were kept in the table because they had different optimized MS parameters, Demographic and clinical information for the patients and healthy controls included in the CRC study are presented in Table 3. The concentrations of spiked U-¹³C¹⁵N-amino acids in the 1:4 dilution sample are listed in Table 4.

The general HILICIRP-LC strategy for GOT-MS is illustrated in FIG. 6. Typical RP C18-SIMs in the m/z range of 100-190 from a pooled serum sample under positive and negative ion detection modes in GOT-MS are presented in FIG. 7. FIG. 8A presents the intraday (n=3) and interday (n=3×3 consecutive days) CVs for amino acids detected by GOT-MS, while FIG. 8B presents the corresponding CVs of amino acids detected by GOT-MS, after normalization to the corresponding isotope labeled (U-¹³C′⁵N-) internal standards. FIG. 8C shows the linearity (R2) of amino acids from the 5 dilution samples in GOT-MS. lle and Leu were integrated together since they had the same MRMs and baseline separation between them was not observed. The intraday (n=3) and interday (n=3×3 consecutive days) CVs of amino acids detected by Q-TOF-MS, before and after normalization to the corresponding isotope labeled (U-¹³C¹⁵N-) internal standards, are presented in FIGS. 9A and 9B, respectively. FIG. 9C shows the linearity (R2) of amino acids detected by Q-TOF-MS using the 5 dilution samples. Ile and Leu were integrated together since they had the same MRMs and baseline separation between them was not observed. Cysteine was not detectable in these samples using Q-TOF. U-¹³C¹⁵N-cysteine, U-¹³C¹⁵N-serine, and U-¹³C¹⁵N-histidine were not detectable in these samples using Q-TOF. R² for Arginine was very small.

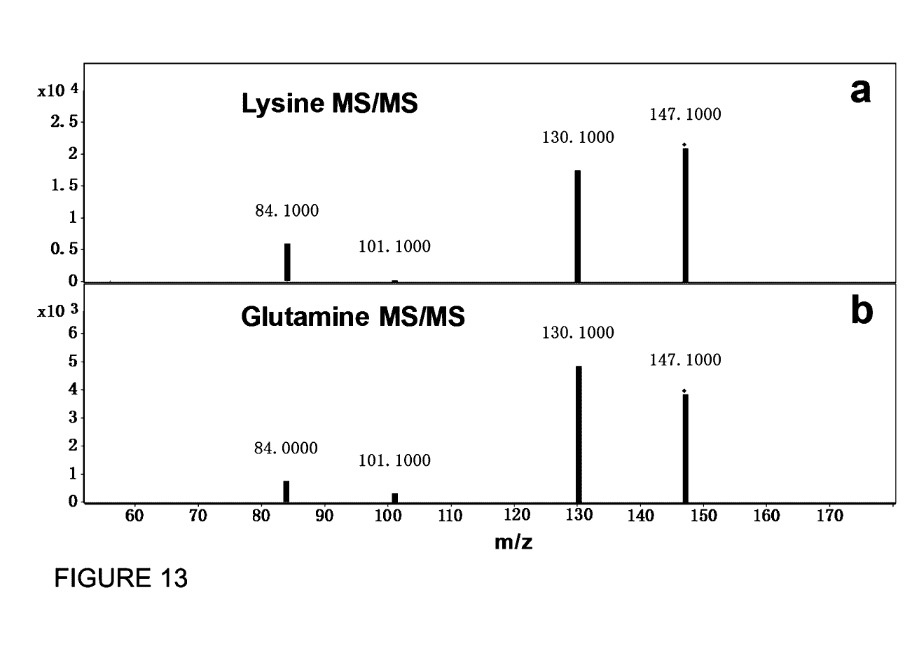

A flow chart of GOT-MS-based metabolomics for CRC diaanosis in the study described in Example 1 is presented in FIG. 10. FIG. 11 shows the PCA score plot (PC1 vs. PC2) for the 155 GOT-MS MRMs collected from CRC and healthy control samples. FIG. 12A is a SIM scan of m/z 147 in GOT-MS, while FIG. 12B is the MS/MS spectrum targeting the ions with m/z 147 at 4.4 min in 12A. FIG. 12C is the MRM scan (147→130.1) of glutamine standard, and FIG. 12D the MRM scan (147→130.1) of lysine standard. FIG. 13A shows the MS/MS spectrum of lysine standard, and 13B the MS/MS spectrum of glutamine standard.

Table 2: GOT-MS MRMs

See table submitted as an ASCII text file as UW58USU1_TABLE2.

TABLE 3
Demographic and clinical information

| | Healthy Controls | | CRC Patients | |
|---|---|---|---|---|
| Subjects | 20 | | 18 | |
| Age, median (range) | 58 | (21-78) | 52 | (27-76) |
| BMI^{a}, median (range) | 27.4 | (20.1-40.0) | 27.0 | (20.9-32.3) |

| | | | |
|---|---|---|---|
| Gender | | | |
| | Male | 11 | 12 |
| | Female | 9 | 6 |
| Stage | | | |
| | I/II | — | 8 |
| | III | — | 7 |
| | IV | — | 3 |
| Ethnicity | | | |
| | Caucasian | 18 | 8 |
| | African American | — | 1 |
| | NA | 2 | 9 |

^{a}1 control and 9 CRC samples do not have BMI data.

TABLE 4
Concentrations of spiked U¹³-C¹⁵N-amino acids

| U-¹³C¹⁵N-Amino Acids | Concentration (μM) |
|---|---|
| U-¹³C¹⁵N-Gly | 87.6 |
| U-¹³C¹⁵N-Ala | 93.2 |
| U-¹³C¹⁵N-Ser | 39.2 |
| U-¹³C¹⁵N-Pro | 58.6 |
| U-¹³C¹⁵N-Val | 28.8 |
| U-¹³C¹⁵N-Thr | 32.5 |
| U-¹³C¹⁵N-Leu | 82.3 |
| U-¹³C¹⁵N-Ile | 16.0 |
| U-¹³C¹⁵N-Asn | 67.3 |
| U-¹³C¹⁵N-Asp | 68.2 |
| U-¹³C¹⁵N-Gln | 60.7 |
| U-¹³C¹⁵N-Lys | 93.5 |
| U-¹³C¹⁵N-Glu | 64.7 |
| U-¹³C¹⁵N-Met | 14.2 |
| U-¹³C¹⁵N-His | 6.7 |
| U-¹³C¹⁵N-Phe | 11.2 |
| U-¹³C¹⁵N-Arg | 56.4 |
| U-¹³C¹⁵N-Tyr | 7.1 |
| U-¹³C¹⁵N-Trp | 16.3 |
| U-¹³C¹⁵N-Cys | 5.4 |

### REFERENCES

(1) Lindon, J. C.; Nicholson, J. K. Expert Opin. Drug Metab. Toxicol. 2014, 10, 915-919.

(2) Griffin, J. L.; Atherton, H.; Shockcor, J.; Atzori, L. Nat. Rev. Cardiol. 2011, 8, 630-643.

(3) Gowda, G. A. N.; Raftery, D. Curr. Metabolomics 2013, 1, 227-240.

(4) Halama, A.; Riesen, N.; Moller, G.; de Angelis, M. H.; Adamski, J. J. Intern. Med. 2013, 274, 425-439.

(5) Scalbert, A.; Brennan, L.; Fiehn, O.; Hankemeier, T.; Kristal, B. S.; van Ommen, B.; Pujos-Guillot, E.; Verheij, E.; Wishart, D.; Wopereis, S. Metabolomics 2009, 5, 435-458.

(6) Gu, H.; Gowda, G. A. N.; Raftery, D. Future Oncol. 2012, 8, 1207-1210.

(7) Dunn, W. B.; Broadhurst, D. I.; Atherton, H. J.; Goodacre, R.; Griffin, J. L. Chem. Soc. Rev. 2011, 40, 387-426.

(8) Bain, J. R.; Stevens, R. D.; Wenner, B. R.; Ilkayeva, O.; Muoio, D. M.; Newgard, C. B. Diabetes 2009, 58, 2429-2443.

(9) Fiehn, O. Plant Mol. Biol. 2002, 48, 155-171.

(10) Heiden, M. G. V.; Cantley, L. C.; Thompson, C. B. Science 2009, 324, 1029-1033.

(11) Carroll, P. A.; Diolaiti, D.; McFerrin, L.; Gu, H.; Djukovic, D.; Du, J.; Cheng, P. F.; Anderson, S.; Ulrich, M.; Hurley, J. B.; Raftery, D.; Ayer, D. E.; Eisenman, R. N. Cancer Cell 2015, 27, 271-285.

(12) Griffin, J. L. Curr. Opin. Chem. Biol. 2003, 7, 648-654.

(13) Kumar, B.; Prakash, A.; Ruhela, R. K.; Medhi, B. Pharmacol. Rep. 2014, 66, 956-963.

(14) Roberts, L. D.; Gerszten, R. E. Cell Metab. 2013, 18, 43-50.

(15) Zhu, J.; Djukovic, D.; Deng, L.; Gu, H.; Himmati, F.; Chiorean, E. G.; Raftery, D. J. Proteome Res. 2014, 13, 4120-4130.

(16) Wang, Z. N.; Klipfell, E.; Bennett, B. J.; Koeth, R.; Levison, B. S.; Dugar, B.; Feldstein, A. E.; Britt, E. B.; Fu, X. M.; Chung, Y. M.; Wu, Y. P.; Schauer, P.; Smith, J. D.; Allayee, H.; Tang, W. H. W.; DiDonato, J. A.; Lusis, A. J.; Hazen, S. L. Nature 2011, 472, 57-U82.

(17) Nicholson, J. K.; Holmes, E.; Kinross, J. M.; Darzi, A. W.; Takats, Z.; Lindon, J. C. Nature 2012, 491, 384-392.

(18) Bundy, J. G.; Davey, M. P.; Viant, M. R. Metabolomics 2009, 5, 3-21.

(19) Rezzi, S.; Ramadan, Z.; Fay, L. B.; Kochhar, S. J. Proteome Res. 2007, 6, 513-525.

(20) Lei, Z. T.; Huhman, D. V.; Sumner, L. W. J. Biol. Chem. 2011, 286, 25435-25442.

(21) Dettmer, K.; Aronov, P. A.; Hammock, B. D. Mass Spectrom. Rev. 2007, 26, 51-78.

(22) Raftery, D (Editor) Mass Spectrometry in Metabolomics-Methods and Protocols; Springer: New York, 2014.

(23) Griffiths, W. J.; Koal, T.; Wang, Y. Q.; Kohl, M.; Enot, D. P.; Deigner, H. P. Angew. Chem. 2010, 49, 5426-5445.

(24) Theodoridis, G.; Gika, H. G.; Wilson, I. D. TRAC-Trend Anal. Chem. 2008, 27, 251-260.

(25) Gu, H.; Du, J.; Neto, F. C.; Carroll, P. A.; Turner, S. J.; Chiorean, E. G.; Eisenman, R. N.; Raftery, D. Analyst 2015, 140, 2726-2734.

(26) Qi, Y. P.; Gu, H. W.; Song, Y. L.; Dong, X.; Liu, A. J.; Lou, Z. Y.; Fan, G. R.; Chai, Y. F. Evid. Based Complement. Alternat. Med. 2013, 438680.

(27) Yao, W. F.; Gu, H. W.; Zhu, J. J.; Barding, G.; Cheng, H. B.; Bao, B. H.; Zhang, L.; Ding, A. W.; Li, W. Anal. Bioanal. Chem. 2014, 406, 7367-7378.

(28) Liu, X. J.; Ser, Z.; Locasale, J. W. Anal. Chem. 2014, 86, 2175-2184.

(29) Dai, D. F.; Karunadharma, P. P.; Chiao, Y. A.; Basisty, N.; Crispin, D.; Hsieh, E. J.; Chen, T.; Gu, H. W.; Djukovic, D.; Raftery, D.; Beyer, R. P.; MacCoss, M. J.; Rabinovitch, P. S. Aging Cell 2014, 13, 529-539.

(30) Reyes, N. L.; Banks, G. B.; Tsang, M.; Margineantu, D.; Gu, H.; Djukovic, D.; Chan, J.; Torres, M.; Liggitt, H. D.; Hirenallur-S, D. K.; Hockenbery, D. M.; Rafteryd, D.; Iritania, B. M. Proc. Natl. Acad. Sci. U. S. A. 2014, 112, 424-429.

(31) Wei, R.; Li, G. D.; Seymour, A. B. Anal. Chem. 2010, 82, 5527-5533.

(32) Yuan, M.; Breitkopf, S. B.; Yang, X. M.; Asara, J. M. Nat. Protoc. 2012, 7, 872-881.

(33) Jain, M.; Nilsson, R.; Sharma, S.; Madhusudhan, N.; Kitami, T.; Souza, A. L.; Kafri, R.; Kirschner, M. W.; Clish, C. B.; Mootha, V. K. Science 2012, 336, 1040-1044.

(34) Mayers, J. R.; Wu, C.; Clish, C. B.; Kraft, P.; Torrence, M. E.; Fiske, B. P.; Yuan, C.; Bao, Y.; Townsend, M. K.; Tworoger, S. S.; Davidson, S. M.; Papagiannakopoulos, T.; Yang, A.; Dayton, T. L.; Ogino, S.; Stampfer, M. J.; Giovannucci, E. L.; Qian, Z. R.; Rubinson, D. A.; Ma, J.; Sesso, H. D.; Gaziano, J. M.; Cochrane, B. B.; Liu, S. M.; Wactawski-Wende, J.; Manson, J. E.; Pollak, M. N.; Kimmelman, A. C.; Souza, A.; Pierce, K.; Wang, T. J.; Gerszten, R. E.; Fuchs, C. S.; Vander Heiden, M. G.; Wolpin, B. M. Nat. Med. 2014, 20, 1193-1198.

(35) Chen, S. L.; Kong, H. W.; Lu, X.; Li, Y.; Yin, P. Y.; Zeng, Z. D.; Xu, G. W. Anal. Chem. 2013, 85, 8326-8333.

(36) Shao, Y.; Zhu, B.; Zheng, R.; Zhao, X.; Yin, P.; Lu, X.; Jiao, B.; Xu, G.; Yao, Z. J. Proteome Res. 2015, 14, 906-916.

(37) Nikolskiy, I.; Siuzdak, G.; Patti, G. J. Bioinformatics 2015, pii: btv085.

(38) Gillet, L. C.; Navarro, P.; Tate, S.; Rost, H.; Selevsek, N.; Reiter, L.; Bonner, R.; Aebersold, R. Mol. Cell. Proteomics 2012, 11.

(39) Zhu, X. C.; Chen, Y. P.; Subramanian, R. Anal. Chem. 2014, 86, 1202-1209.

(40) Chen, C.; Deng, L.; Wei, S.; Gowda, G. A. N.; Gu, H.; Chiorean, E. G.; Zaid, M. A.; Harrison, M. L.; Pekny, J. F.; Loehrer, P. J.; Zhang, D.; Zhang, M.; Raftery, D. J. Proteome Res. 2015, 14, 2492-2499.

(41) Siegel, R. L.; Miller, K. D.; Jemal, A. CA-Cancer J. Clin. 2015, 65, 5-29.

(42) Chan, E. C. Y.; Koh, P. K.; Mal, M.; Cheah, P. Y.; Eu, K. W.; Backshall, A.; Cavill, R.; Nicholson, J. K.; Keun, H. C. J. Proteome Res. 2009, 8, 352-361.

(43) Piotto, M.; Moussallieh, F. M.; Dillmann, B.; Imperiale, A.; Neuville, A.; Brigand, C.; Bellocq, J. P.; Elbayed, K.; Namer, I. J. Metabolomics 2009, 5, 292-301.

(44) Denkert, C.; Budczies, J.; Weichert, W.; Wohlgemuth, G.; Scholz, M.; Kind, T.; Niesporek, S.; Noske, A.; Buckendahl, A.; Dietel, M.; Fiehn, O. Mol. Cancer 2008, 7, 72.

(45) Lean, C. L.; Newland, R. C.; Ende, D. A.; Bokey, E. L.; Smith, I. C. P.; Mountford, C. E. Magn. Reson. Med. 1993, 30, 525-533.

(46) Qiu, Y. P.; Cai, G. X.; Su, M. M.; Chen, T. L.; Zheng, X. J.; Xu, Y.; Ni, Y.; Zhao, A. H.; Xu, L. X.; Cai, S. J.; Jia, W. J. Proteome Res. 2009, 8, 4844-4850.

(47) Ritchie, S. A.; Ahiahonu, P. W. K.; Jayasinghe, D.; Heath, D.; Liu, J.; Lu, Y. S.; Jin, W.; Kavianpour, A.; Yamazaki, Y.; Khan, A. M.; Hossain, M.; Su-Myat, K. K.; Wood, P. L.; Krenitsky, K.; Takemasa, I.; Miyake, M.; Sekimoto, M.; Monden, M.; Matsubara, H.; Nomura, F.; Goodenowe, D. B. Bmc Medicine 2010, 8, 13.

(48) Leichtle, A. B.; Nuoffer, J. M.; Ceglarek, U.; Kase, J.; Conrad, T.; Witzigmann, H.; Thiery, J.; Fiedler, G. M. Metabolomics 2012, 8, 643-653.

(49) Tan, B. B.; Qiu, Y. P.; Zou, X.; Chen, T. L.; Xie, G. X.; Cheng, Y.; Dong, T. T.; Zhao, L. J.; Feng, B.; Hu, X. F.; Xu, L. X.; Zhao, A. H.; Zhang, M. H.; Cai, G. X.; Cai, S. J.; Zhou, Z. X.; Zheng, M. H.; Zhang, Y.; Jia, W. J. Proteome Res. 2013, 12, 3000-3009.

(50) Qiu, Y. P.; Cai, G. X.; Su, M. M.; Chen, T. L.; Liu, Y. M.; Xu, Y.; Ni, Y.; Zhao, A. H.; Cai, S. J.; Xu, L. X.; Jia, W. J. Proteome Res. 2010, 9, 1627-1634.

(51) Monleon, D.; Morales, J. M.; Barrasa, A.; Lopez, J. A.; Vazquez, C.; Celda, B. NMR Biomed. 2009, 22, 342-348.

(52) http://metlin.scripps.edu/.

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A method for subjecting a mixture of molecules to a global search of precursor and product ions with a single mass spectrometer (MS), the method comprising:
(a) performing selected ion monitoring (SIM) incremental scanning or incremental MS scanning using a triple quadrupole (QQQ) mass spectrometer to obtain globally selected precursor ions in a certain mass range from the mixture;
(b) carrying out tandem mass spectrometry (MS/MS) scanning with incremental collision energy (CE) for each selected precursor ion to profile product ions, thereby producing selected pairs of precursor and product ions; and
(c) optimizing the selected pairs of precursor and product ions for different MS parameters.

2. A method of globally detecting metabolites within a complex mixture, comprising performing the method of claim 1.

3. The method of claim 2, wherein over 1,000 multiple reaction monitoring (MRM) signals are detected.

4. The method of claim 1, wherein liquid chromatography is performed on the mixture prior to step (a).

5. The method of claim 4, wherein the liquid chromatography is HILIC or reverse phase liquid chromatography (RPLC).

6. The method of claim 5, wherein the HILIC or RPLC is used to separate aqueous or lipid metabolites in the mixture.

7. The method of claim 1, wherein the SIM incremental scanning or incremental MS scanning of step (a) is performed in the mass range of 40-2000 Da.

8. The method of claim 1, wherein the CE of step (b) is of a voltage of 5 V to 250 V.

9. The method of claim 8, wherein the CE of step (b) is of a voltage selected from the group consisting of 5 V, 15 V, and 25 V.

10. The method of claim 1, wherein the mixture comprises a biofluid mixture, tissue, or cellular mixture.

11. The method of claim 10, wherein the biofluid mixture is a serum sample.

12. The method of claim 10, wherein the biofluid mixture is a urine sample.

13. A Globally Optimized Targeted Mass Spectrometry (GOT-MS) system comprising:
(a) a component adapted to receive injections of sample;
(b) a scanning component, wherein the scanning comprises selected ion monitoring (SIM) incremental scanning or incremental MS scanning;
(c) a triple quadrupole spectrometer adapted to perform tandem mass spectrometry (MS/MS) and multiple reaction monitoring (MRM); and
(d) a detection means adapted to maximize the number of MRMs that can be detected with each injection of sample.

14. The GOT-MS system of claim 13, wherein the component of (a) is a hydrophilic interaction liquid chromatography (HILIC) or reverse phase (RP) liquid chromatography component.

15. The GOT-MS system of claim 13, wherein the scanning component scans in a range of 40 to 2000 Daltons.

16. The GOT-MS system of claim 13, wherein the MS/MS employs a collision energy of 5 to 250 Volts.

17. The GOT-MS system of claim 13, wherein each of (a) to (d) is contained within a single instrument.

18. A method of Globally Optimized Targeted Mass Spectrometry (GOT-MS) comprising:
(a) preparing an extract of metabolites from a biological sample; and
(b) injecting the aqueous extract into the GOT-MS system of claim 12.

19. The method of claim 18, wherein the extract is an aqueous extract.

20. The method of claim 18, wherein the extract is a lipid extract.
